(19) United States
(12) Patent Application Publication
(10) Pub. No.: US 2023/0372936 A1
(43) Pub. Date: Nov. 23, 2023

(54) METHODS FOR PURIFYING T CELLS

(71) Applicant: CARINA BIOTECH PTY LTD, Mawson Lakes, South Australia (AU)

(72) Inventors: Mona Tarek Mohamed Hassan ELSEMARY,, South Australia (AU); Louise Elizabeth SMITH, South Australia (AU); Michelle Frances MARITZ, South Australia (AU); Benjamin THIERRY, South Australia (AU)

(21) Appl. No.: 18/247,539

(22) PCT Filed: Sep. 30, 2021
(86) PCT No.: PCT/AU2021/051145

§ 371 Date: Mar. 31, 2023

(30) Foreign Application Priority Data
Oct. 1, 2020 (AU) 2020903560
Mar. 10, 2021 (AU) 2021900687

(51) Int. Cl. B01L 3/00

(52) U.S. Cl. CPC B01L 3/502753; B01L 2200/0636; B01L 2200/0652; B01L 2300/088

(57) ABSTRACT

The present disclosure relates to methods of purifying T cells, to T cells and T cell products produced by the methods, and use of the cells and products for therapy. In certain embodiments, the present disclosures provides a method of purifying T cells. The method comprises subjecting a medium comprising the T cells to inertial microfluidic fractionation and obtaining a fraction comprising purified T cells.

### PRIORITY CLAIM

This application claims priority to Australian Provisional Patent Application 2020903560 Australia filed on 1 Oct. 2020 and Australian Provisional Patent Application 2021900687 filed on 10 Mar. 2021, the contents of both which are hereby incorporated by reference.

### FIELD

The present disclosure relates to methods of purifying T cells, T cells and T cell products produced by the methods, and use of the cells and products for therapy.

### BACKGROUND

In recent years a variety of therapies have been developed using T cells. Such therapies include adoptive T cell transfer and Chimeric Antigen Receptor T cell (CAR-T cell) therapy.

CAR-T cell therapy in particular has proven to be a transformative cancer therapy. Recently the FDA has approved two CAR-T cells based drugs treatment for B cell acute lymphoblastic leukemia, and a number of other CAR-T cell formulations are at the investigational stage, with over 500 clinical trials underway targeting various cancers.

However, the CAR-T cell manufacturing process is associated with a number of significant barriers and a high cost of goods, preventing the full potential of the therapy to be reached. Problems associated with the manufacturing of CAR-T cells for therapeutic use include significant variability of the starting cellular material due to either patients' cellular inconsistency, the use of different cell collection/enrichment devices providing inconsistency in the products, and the presence of undesired constituents in media, all of which can affect the use of the cells.

In this regard, the presence of non-viable cells and debris is an inevitable consequence of the complex CAR-T cells manufacturing process, and which has significant impact on the CAR-T cell industry. The FDA has set a strict viability specification for CAR-T products, and in some cases the minimal acceptable viability is 80% in a commercial product and 70% in the context of clinical trials. It is also noteworthy that CAR-T cell manufacturing companies cannot charge for an out of specification product, incurring significant manufacture costs. Importantly, while debris can usually be removed by centrifugation, non-viable cells are harder to remove, especially at the point-of-care prior to administration to patients.

Cryoprotectants such as DMSO are also typically used for the freezing and storage of CAR T cell products. However, it has been shown that the presence of cryoprotectants can cause severe allergic reactions and toxic side-effects in patients. It is therefore preferable to remove or at least reduce their concentration in the final product. Current commercial CAR-T cell products contain significant amounts of DMSO.

Accordingly, there is a need for new methods for purifying T cells which can be used to improve the manufacturability of T cell products.

### SUMMARY

The present disclosure relates to methods of purifying T cells, to T cells and T cell products produced by the methods, and use of the cells and products for therapy.

Certain embodiments of the present disclosure provide a method of purifying T cells, the method comprising subjecting a medium comprising the T cells to inertial microfluidic fractionation and obtaining a fraction comprising purified T cells.

Certain embodiments of the present disclosure provide a method of purifying T cells, the method comprising purifying the T cells from non-viable cells and/or one or more undesired constituents present in a medium by subjecting the medium to inertial microfluidic fractionation and obtaining a fraction comprising purified T cells.

Certain embodiments of the present disclosure provide T cells purified by a method as described herein.

Certain embodiments of the present disclosure provide use of T cells purified by a method as described herein for therapy.

Certain embodiments of the present disclosure provide a medicament comprising T cells purified by a method as described herein.

Certain embodiments of the present disclosure provide a method of producing a T cell product, the method comprising subjecting a medium comprising the T cells to inertial microfluidic fractionation and obtaining a fraction of the medium comprising purified T cells.

Certain embodiments of the present disclosure provide a T cell product produced by a method as described herein.

Certain embodiments of the present disclosure provide use of a T cell product produced by a method as described herein for therapy.

Certain embodiment of the present disclosure provide a medicament comprising a T cell product produced by a method as described herein.

Certain embodiments of the present disclosure provide a T cell product comprising at least 50% viable T cells, the T cell product produced by inertial microfluidic fractionation.

Certain embodiments of the present disclosure provide a method of depleting non-viable cells from a mixture comprising T cells, the method comprising subjecting the mixture comprising the T cells to inertial microfluidic fractionation and obtaining a fraction enriched for T cells and depleted in non-viable cells.

Certain embodiments of the present disclosure a method of depleting one or more undesired constituents from a T cell mixture, the method comprising subjecting a mixture comprising T cells and one or more undesired constituents to inertial microfluidic fractionation and obtaining a fraction comprising viable T cells and depleted in one or more of the undesired constituents.

Certain embodiments of the present disclosure provide a method of improving transfection characteristics of a T cell population, the method comprising purifying T cells present in a medium by subjecting the medium to inertial microfluidic fractionation and purifying the T cells present in a fraction of the medium, thereby improving the transfection characteristics of the T cell population.

Certain embodiments of the present disclosure provide an inertial microfluidic fractionation device comprising at least two inlets and a plurality of outlets, the device comprising a channel having a channel height of 50 to 150 μm.

Certain embodiments of the present disclosure provide use of a device as described herein for purifying T cells.

Other embodiments are described herein.

### BRIEF DESCRIPTION OF THE FIGURES

Certain embodiments are illustrated by the following figures. It is to be understood that the following description is for the purpose of describing particular embodiments only and is not intended to be limiting with respect to the description.

FIG. 1 shows a schematic representation of the design and operation of the inertial microfluidic device. Viable cells are collected in the innermost outlet 6 while dead cells are collected in outlets 2-5 and debris in outlet 1.

FIG. 2 shows size distribution of viable and dead CAR-T cells. Cells were analysed using imaging flow cytometry and the size distributions were calculated using an adaptive erode mask (M04 CH04 77) on the AMNIS software and plotted using FlowJo.

FIG. 3 shows inertial microfluidic enrichment of viable CAR-T cells suspended in PBS+1% FBS. The percentage (indicated by *) of viable cells increased significantly for the 3 tested CAR-T cell batches (p<0.05).

FIG. 4 shows enrichment of viable CAR-T cells suspended in Cryostor and reformulated in plasmalyte+5% BSA. The percentage viability (indicated by *) increased significantly at p<0.05 for CAR-T (n=4).

FIG. 5 shows the effect of inertial microfluidics on the depletion of DMSO from cryopreserved T-cells in Cryostor. a) Identification of the DMSO and BSA peaks using capillary electrophoresis b) Percentage of DMSO before and after processing in the device with inlet flow rate ratio of 1:10 (cell in Cryostor: plasmalyte+5% BSA).

FIG. 6 shows effect of inertial microfluidic processing on the percentage transduction in CD4 and CD8 T-cells. Percentages of (a) CD4 and CD8 T-cells (b) percentage transduced cells in the CD4 and CD8 T-cells with and without inertial microfluidic processing after 12 days of expansion.

FIG. 7 shows the effect of inertial microfluidic processing on the frequency of different phenotypes of CD4 and CD8 CAR-T cells. Percentages of Naïve, central memory (CM), effector memory (EM) and terminally differentiated effector cells (EMRA) for a) CD4 and b) CD8 CAR-T cells c) Percentage of CD4 and CD8 stem cell phenotype d) Number of CD4 T-regulatory and CD8 T-suppressor cells. (n=5, * p<0.05, ** p<0.005, ** p<0.0005).

FIG. 8 shows effects of inertial microfluidic processing on T cell proliferation and cytotoxicity. a) Proliferative Index compared between device separated and non-device separated cells at 72 and 168 hr for both CAR-T and untransduced cells (n=2). c) Percentage specific lysis for CAR-T and untransduced control T cells with and without inertial microfluidic processing (n=3).

FIG. 9 shows DMSO measurements by UV-Vis spectrophotometry using the Thermo Evolution 201 UV-VIS spectrophotometer a) Screening for the Amax of DMSO b) Calibration curve of DMSO in PBS determined

FIG. 10 shows capillary electrophoresis of DMSO and Cryostor in the presence and absence of plasmlyte+5% BSA. a) electropherograms of DMSO, DMSO in plasmalyte+5% BSA, Cryostor and Cryostor in plasmalyte+5% BSA b) Calibration curves of i) DMSO ii) Cryostor ii) DMSO in plasmalyte+5% BSA iii) Cryostor iv) Cryostor in plasmalyte+5% BSA. The curves were measured at wavelength of 209 nm after capillary electrophoretic separation. Each point was repeated three times and read three times on the instrument. The peak area was divided by the migration time to correct for differences in electrophoretic mobility.

FIG. 11 shows effect of different flow rate and flow rate combinations of DMSO containing cell Inlet: PBS Inlet on a. DMSO concentration in the various outlets (experiment done without cells) b. Raji cell number recovery in each outlet c. DMSO concentration in each outlet (experiment done with Raji cells) d. A549 cell number recovery in each outlet e. DMSO concentration in each outlet (experiment done with A549 cells).

FIG. 12 shows gating strategy and controls for live and dead cell staining a) Gating for viable vs. dead cells on the imaging flow cytometer b) Positive permeabilized PI control and negative control (CD3 stained only).

FIG. 13 shows size distribution of Batch 3 CAR-T and untransduced cells (UN) purified in the device. Representative histograms of size distribution of viable and dead cells before and after device separation in the collection outlets (Cell collection outlet 6 and the dead cell waste outlet 1-5).

FIG. 14 shows optimization of the recovery of viable cells and depletion of dead cells for CAR-T cells in Cryostor and reformulation in plasmalyte+5% BSA. Different device configuration, flow rates and ratios were tested: 1) Flow rate ratio 1:1, long outlet, flow rate 1 ml/min 2) Flow rate ratio 1:1, short outlet, flow rate 1 mL/min, 3) Flow rate ratio 1:1, long outlet, flow rate 1.5 mL/min, 4) Flow rate ratio 1:3, short outlet, flow rate 1 ml/min, 5) Flow rate ratio 1:10, long outlet, flow rate 1 mL/min, 6) PBS+1% FBS+10% DMSO, Flow rate ratio 1:1, long outlet, flow rate 1 ml/min (the optimised parameters for cells resuspended in PBS+1% FBS+10% DMSO).

FIG. 15 shows inertial microfluidic enrichment of viable untransduced T-cells. Number of viable and dead cells was counted by Trypan Blue staining before and after purification in the device a) suspended in PBS+1% FBS b) using the new inlet flow rate ratio of 1:10 (cell suspension in Cryostor: plasmalyte+5% BSA). The percentage (indicated by *) of viable cells increased significantly for the tested untransduced T-cell batches (p<0.05).

FIG. 16 shows gating strategy used for detection of the percentage transduction and percentage CD4 and CD8 T-cells before and after device separation of CAR-T cell product. a) Gating Strategy Used b) Unstained, FMOs and Positive Controls for CD4 and CD8 c) Unstained, FMOs and Positive Controls for EGFR.

FIG. 17 shows gating strategy used for detection of the percentage of different CD4 cell phenotypes before and after device separation of CAR-T cell product. a) Gating Strategy Used. Unstained, FMOs and Positive Controls for b) CD4 c) CCR7 and CD45RO d) CD45RA.

FIG. 18 shows gating strategy used for detection of the percentage of different CD8 cell phenotypes before and after device separation of CAR-T cell product. a) Gating Strategy Used. Unstained, FMOs and Positive Controls for b) CD8 c) CCR7 and CD45RO d) CD45RA.

FIG. 19 shows the effect of inertial microfluidic processing on the frequency of different phenotypes of CD4 and CD8 untransduced T cells. Percentages of Naïve, central memory (CM), effector memory (EM) and terminally differentiated effector cells (EMRA) for a) CD4 and b) CD8 CAR-T cells c) Percentage of CD4 and CD8 stem cell phenotype d) Number of CD4 T-regulatory and CD8 T-suppressor cells. (n=5, * p<0.05, ** p<0.005).

### DETAILED DESCRIPTION

The present disclosure relates to methods of purifying T cells, to T cells and T cell products produced by the methods, and use of the cells and products for therapy.

The present disclosure is based on the demonstration that inertial microfluidic fractionation devices efficiently remove non-viable cells from CAR-T cells while at the same time reducing the cryoprotectant DMSO concentration in the cell media. The enrichment process does not impact the proliferation rates and cytotoxicity of the CAR-T cells, and improves the phenotypic composition of the product.

Certain embodiments of the present disclosure provide a method of purifying T cells by inertial microfluidic fractionation.

In certain embodiments, the present disclosure provides a method of purifying T cells, the method comprising subjecting a medium comprising the T cells to inertial microfluidic fractionation and obtaining a fraction comprising purified T cells.

The term “purifying” and related terms such as “purify” or “purified”, as used herein refer to increasing or enriching the proportion of one or more particular species, such as cells of a particular type, in a mixture of species (such as other cells).

The term “isolating” and related terms such as “isolate” or “isolated” refer to a process whereby a species, such as a cell, has been separated (partially or completely) from its starting environment.

Methods for inertial microfluidic fractionation are known in the art, or example as described in Zhang et al. (2016) Lab on a Chip 16: 10-34. Devices for inertial microfluidic fractionation may be obtained commercially or produced by a method known in the art.

In certain embodiments, the purification comprises purification utilising Dean vortices.

In certain embodiments, the inertial microfluidic fractionation comprises spiral inertial microfluidic fractionation.

T cells and methods for identifying T cells are known in the art. T cells express the CD3+ surface marker. Examples of T cells include CAR-T cells, memory T cells and central memory T cells.

In certain embodiments, the T cells comprise CD8+ T cells. In certain embodiments, the T cells comprise CD4+ T cells.

In certain embodiments, the T cells comprise human T cells. However, the use of non-human T cells is also contemplated, including their use in veterinary applications of the present disclosure.

In certain embodiments, the T cells comprises one or more of ex vivo T cells, isolated ex vivo T cells, untransduced T cells, transduced T cells, or CAR-T cells.

In certain embodiments, the T cell cells comprise CAR-T cells. Methods for producing CAR-T cells are known in the art.

In certain embodiments, the T cells comprise expanded T cells. In certain embodiments, the T cells comprises cells that have been frozen and thawed.

In certain embodiments, the medium comprising the T cells comprises a cell culture medium, or a minimal medium for holding the cells (eg PBS+BSA). In certain embodiments, the medium comprises a cell culture medium used for expanding cells after thawing. In certain embodiments, the medium comprises a cell expansion medium.

An appropriate fraction comprising the purified T cells may be obtained. In certain embodiments, the method comprises inertial microfluidic spiral fractionation and obtaining a fraction comprising purified T cells.

In certain embodiments, the method comprises use of a device comprising a channel height of approximately 130 μm. In certain embodiments, the method comprises use of a device comprising a channel height of at least than 50 μm. In certain embodiments, the method comprises use of a device comprising a channel height in the range from 50 to 200 μm, 50 to 150, 50 to 100, 100 to 200, 100 to 150, or 150 to 200 μm. Inertial microfluidic fractionation devices comprising the aforementioned characteristics may be obtained commercially or produced by a method known in the art.

In certain embodiments, the method comprises use of an inertial spiral microfluidic fractionation device comprising approximately 8 turns. In certain embodiments, the method comprises use of an inertial spiral microfluidic fractionation device comprising at least 1 turns. In certain embodiments, the method comprises use of an inertial spiral microfluidic fractionation device having 1, 2, 3, 4, 5, 6, 7, 8, 9 or 10 turns. In certain embodiments, the method comprises use of an inertial spiral microfluidic fractionation device comprising 1 to 10 turns. Inertial spiral microfluidic fractionation devices comprising the aforementioned characteristics may be obtained commercially or produced by a method known in the art.

In certain embodiments the inertial microfluidic fractionation comprises an inlet ratio of the medium to a sheath medium of at least 1:1.

In certain embodiments the inertial microfluidic fractionation comprises an inlet ratio of the medium to a sheath medium of at least 1:1, at least 1:2; at least 1:3, at least 1:4, at least 1:5, at least 1:6, at least 1:7, at least 1:8, at least 1:9 or at least 1:10.

In certain embodiments, the inertial microfluidic fractionation comprises an inlet ratio of the medium to the sheath medium of approximately 1:10.

A suitable medium for fractionation may be selected.

In certain embodiments, the inertial microfluidic fractionation utilises a flow rate of at least 1 ml/min. In certain embodiments, the method comprises inertial microfluidic fractionation utilising a flow rate of at least 1 ml/min. Other flow rates are contemplated.

In certain embodiments, the method comprises fractionating or separating the T cells from non-viable cells and/or one or more undesired constituents present in the medium.

In certain embodiments, the non-viable cells comprise non-viable T cells. Methods for identifying non-viable cells are known in the art.

In certain embodiments, the medium comprises 1×10⁵/ml to 1×10⁸/ml T cells. In certain embodiments, the T cell product comprises at least 10⁶ cells, at least 10⁷ cell, or at least 10⁸ cells.

In certain embodiments, the medium comprises at least 1×10⁶/ml T cells. In certain embodiments, the medium comprises 1×10⁶/ml to 5×10⁶/ml, 1×10⁶/ml to 1×10⁷/ml, 1×10⁶/ml to 2×10⁷/ml, 1×10⁶/ml to 3×10⁷/ml, 5×10⁶/ml to 1×10⁷/ml, 5×10⁶/ml to 2×10⁷/ml, 5×10⁶/ml to 3×10⁷/ml, 1×10⁷/ml to 2×10⁷/ml, 1×10⁷/ml to 3×10⁷/ml or

In certain embodiments, the method comprises purification of a medium comprising at least 1×10⁶/ml T cells. In certain embodiments, the method comprises purification of a medium comprising 1×10⁶/ml to 5×10⁶/ml, 1×10⁶/ml to 1×10⁷/ml, 1×10⁶/ml to 2×10⁷/ml, 1×10⁶/ml to 3×10⁷/ml, 5×10⁶/ml to 1×10⁷/ml, 5×10⁶/ml to 2×10⁷/ml, 5×10⁶/ml to 3×10⁷/ml, 1×10⁷/ml to 2×10⁷/ml, 1×10⁷/ml to 3×10⁷/ml or 2×10⁷/ml to 3×10⁷/ml T cells.

In certain embodiments, the method comprises recovery of at least 50% of viable T cells present in the medium.

In certain embodiments, the method increases viability of the T cells by at least 10%, at least 20%, at least 25%, at least 30%, at least 35%, at least 40%, at least 45%, at least 50%, at least 55%, at least 60%, at least 65%, at least 70%, at least 75%, or at least 80%.

In certain embodiments, the method comprises fractionating or separating the T cells from non-viable cells and/or one or more undesired constituents present in the medium.

In certain embodiments, the non-viable cells comprise non-viable T cells.

In certain embodiments, the method depletes greater than 40%, greater than 50%, greater than 60%, greater than 70%, or greater than 80% of non-viable cells present in the medium.

In certain embodiments, the method depletes 40% to 80%, 40% to 70%, 40% to 60%, 40% to 50%, 50% to 80%, 50% to 70%, 50% to 60%, 60% to 80%, 60% to 70%, or 70% to 80% of non-viable cells present in the medium.

In certain embodiments, the purified T cells comprise less than 20%, less than 15%, or less than 10% non-viable cells.

In certain embodiments, the method further depletes at least 10%, 20%, 30%, 40% or 50% of T-regulatory and/or T-suppressor cells present in the medium.

In certain embodiments, the one or more undesired constituents comprise cell debris.

In certain embodiments, the one or more undesired constituents comprise a constituent present in a medium used for thawing and/or expansion of cells, such as a cryoprotectant. Examples of cryoprotectants include DMSO, ethylene glycol, Propylene glycol, 2-methyl-2,4-pentanediol (MPD), trehalose, formamide and other commercially available cryoprotectants as Cryostor.

In certain embodiments, the one or more undesired constituents comprises DMSO.

In certain embodiments, the concentration of one or more undesired constituents in the medium is reduced by at least 10%, at least 20%, at least 30% or at least 40%. In certain embodiments, the method comprises reducing the concentration of one or more undesired constituents in the medium by at least 40%.

In certain embodiments, the concentration of the one or more undesired constituents is reduced to less than 2%. In certain embodiments, the concentration of the one or more undesired constituents is reduced to less than 1%.

In certain embodiments, the purification of the T cells does not substantially impair the proliferation capacity of the purified cells. Methods for assessing the proliferation capacity of cells are known in the art.

In certain embodiments, the method is used to enrich T cells, to purify T cells from non-viable cells and/or one or more undesired constituents, to manufacture T cells for therapeutic use, to improve a characteristic of the T cells (such as transfectability), or to produce T cells or a T cell product suitable for therapy.

In certain embodiments, the present disclosure provides a method of purifying T cells, the method comprising purifying the T cells from non-viable cells and/or one or more undesired constituents present in a medium by subjecting the medium to inertial microfluidic fractionation and obtaining a fraction comprising purified T cells.

Certain embodiments of the present disclosure provide T cells purified by a method as described herein.

Characteristics of T cells purified in such a manner are described herein.

In certain embodiments, the T cells comprise a reduced concentration of non-viable cells as compared to the originating input T cells.

In certain embodiments, the T cells comprise a reduced concentration of one or more undesired constituents as compared to the originating input medium.

Certain embodiments of the present disclosure provide use of purified T cells as described herein for therapy.

Examples of therapies include immunotherapies, adoptive cell therapies, and CAR-T cell therapies for treating cancers. Methods for using T cells in the aforementioned therapies are known in the art.

Certain embodiments of the present disclosure provide a medicament comprising purified T cells as described herein.

Methods for formulating T cells into a medicament for therapy are known in the art. For example, a composition comprising Lactated Ringers solution with suspended T cells (eg 1×10⁵ to 1×10⁹ cells) may be used.

Certain embodiments of the present disclosure provide a method of producing a T cell product.

In certain embodiments, the present disclosure provides a method of producing a T cell product, the method comprising subjecting a medium comprising T cells to inertial microfluidic fractionation and obtaining a fraction of the medium comprising purified T cells.

Methods for fractionating cells using inertial microfluidic fractionation and obtaining a fraction comprising purified or enriched T cells are as described herein.

In certain embodiments, the method comprises purification utilising Dean vortices.

In certain embodiments, the inertial microfluidic fractionation comprises spiral inertial microfluidic fractionation.

T cells and method for identifying T cells (CD3+) are known in the art.

In certain embodiments, the T cells comprise CAR-T cells, memory T cells, and/or central memory T cells.

In certain embodiments, the T cells comprise CD8+ T cells. In certain embodiments, the T cells comprise CD4+ T cells.

In certain embodiments, the T cells comprise human T cells. The use of non-human T cells is also contemplated.

In certain embodiments, the T cells comprises one or more of ex vivo T cells, isolated ex vivo T cells, untransduced T cells, transduced T cells, or CAR-T cells.

In certain embodiments, the T cell cells comprise CAR-T cells.

In certain embodiments, the T cells comprises cells that have been frozen and thawed.

In certain embodiments, the medium comprising the T cells comprises a cell culture medium or a minimal medium for holding the cells (eg PBS+BSA). In certain embodiments, the medium comprises a cell culture medium used for expanding cells after thawing. In certain embodiments, the medium comprises a cell expansion medium.

An appropriate fraction comprising the purified T cells can be obtained and identified. In certain embodiments, the method comprises inertial microfluidic spiral fractionation and the fraction comprises an earlier fraction than one or more later fractions containing non-viable cells and/or undesired constituents.

In certain embodiments, the method comprises use of a device comprising a channel height of approximately 130 μm. In certain embodiments, the method comprises use of a device comprising a channel height of at least 50 μm. In certain embodiments, the method comprises use of a device comprising a channel height in the range from 50 to 200, 50 to 150, 50 to 100, 100 to 200, 150 to 200, or 100 to 150 μm. Inertial microfluidic fractionation devices comprising the aforementioned characteristics may be obtained commercially or produced by a method known in the art.

In certain embodiments, the method comprises use of an inertial spiral microfluidic fractionation device comprising approximately 8 turns. In certain embodiments, the method comprises use of an inertial spiral microfluidic fractionation device comprising at least 1 turn. In certain embodiments, the method comprises use of an inertial spiral microfluidic fractionation device comprising 1 to 10 turns. In certain embodiments, the method comprises use of an inertial spiral microfluidic fractionation device having 1, 2, 3, 4, 5, 6, 7, 8, 9 or 10 turns. Inertial spiral microfluidic fractionation devices comprising these characteristics may be obtained commercially or produced by a method known in the art, such as photolithography.

In certain embodiments the inertial microfluidic fractionation comprises an inlet ratio of the medium to a sheath medium of at least 1:1.

In certain embodiments the inertial microfluidic fractionation comprises an inlet ratio of the medium to a sheath medium of at least 1:1, at least 1:2; at least 1:3, at least 1:4, at least 1:5, at least 1:6, at least 1:7, at least 1:8, at least 1:9 or at least 1:10. In certain embodiments, the inertial microfluidic fractionation comprises an inlet ratio of the medium to the sheath medium of approximately 1:10.

A suitable medium for fractionation may be selected.

In certain embodiments, the inertial microfluidic fractionation utilises a flow rate of at least 1 ml/min. Other flow rates are contemplated.

In certain embodiments, the method comprises fractionating or separating the T cells from non-viable cells and/or one or more undesired constituents present in the medium.

In certain embodiments, the non-viable cells comprise non-viable T cells. Methods for identifying non-viable cells are known in the art.

In certain embodiments, the medium comprises 1×10⁵/ml to 1×10/ml cells.

In certain embodiments, the medium comprises at least 1×10⁶/ml T cells. In certain embodiments, the medium comprises at least 10⁷ cell, or at least 10⁸ cells.

In certain embodiments, the medium comprises 1×10⁶/ml to 5×10⁶/ml, 1×10⁶/ml to 1×10⁷/ml, 1×10⁶/ml to 2×10⁷/ml, 1×10⁶/ml to 3×10⁷/ml, 5×10⁶/ml to 1×10⁷/ml, 5×10⁶/ml to 2×10⁷/ml, 5×10⁶/ml to 3×10⁷/ml, 1×10⁷/ml to 2×10⁷/ml, 1×10⁷/ml to 3×10⁷/ml or 2×10⁷/ml to 3×10⁷/ml T cells.

In certain embodiments, the method comprises purification of a medium comprising 1×10⁶/ml to 1×10⁸/ml T cells.

In certain embodiments, the medium comprises at least 1×10⁶ T cells. In certain embodiments, the method comprises purification of at least 1×10⁶ input T cells. In certain embodiments, the method comprises fractionation of 1×10⁶ to 5×10⁶, 1×10⁶ to 1×10⁷, 1×10⁶ to 2×10⁷, 1×10⁶ to 3×10⁷, 5×10⁶ to 1×10⁷, 5×10⁶ to 2×10⁷, 5×10⁶ to 3×10⁷, 1×10⁷ to 2×10⁷, 1×10⁷ to 3×10⁷, or 2×10⁷ to 3×10⁷ input T cells.

In certain embodiments, the method comprises recovery of at least 50% of viable T cells present in the medium.

In certain embodiments, the method comprises fractionating or separating the T cells from non-viable cells and/or one or more undesired constituents present in the medium.

In certain embodiments, the non-viable cells comprise non-viable T cells.

In certain embodiments, the method depletes greater than 40%, greater than 50%, greater than 60%, greater than 70%, or greater than 80% of non-viable cells present in the medium.

In certain embodiments, the method depletes 40% to 80%, 40% to 70%, 40% to 60%, 40% to 50%., 50% to 80%, 50% to 70%, 50% to 60%, 60% to 80%, 60% to 70%, or 70% to 80% of non-viable cells present in the medium.

In certain embodiments, the purified T cells comprise less than 20%, less than 15%, less than 10% non-viable cells.

In certain embodiments, the method further depletes at least 10%, at least 20%, at least 30%, at least 40%, or at least 50% of T-regulatory and/or T-suppressor cells present.

In certain embodiments, the one or more undesired constituents comprise cell debris.

In certain embodiments, the one or more undesired constituents comprise a constituent present in a medium, such as a cryoprotectant (eg DMSO or glycerol).

In certain embodiments, the one or more undesired constituents comprises DMSO.

In certain embodiments, the concentration of one or more undesired constituents in the medium is reduced by at least 10%, at least 20%, at least 30%, or at least 40%.

In certain embodiments, the concentration of the one or more undesired constituents is reduced to less than 2%. In certain embodiments, the concentration of the one or more undesired constituents is reduced to less than 1%. In certain embodiments, the one or more undesired constituents comprise DMSO.

In certain embodiments, the purification of the T cells does not substantially impair the proliferation capacity of the purified cells.

Certain embodiments of the present disclosure provide a T cell product produced by a method as described herein.

Certain embodiments of the present disclosure use of a T cell product as described herein for therapy.

Examples of therapies and methods for using T cells for therapeutic purposes are as described herein.

Certain embodiments of the present disclosure provide a medicament comprising a T cell product as described herein.

Medicaments using a T cell product are as described herein.

Certain embodiment of the present disclosure provide a T cell product comprising at least 50% viable T cells, the T cell product produced by inertial microfluidic fractionation.

Methods for producing a T cell product comprising at least 50% viable T cells by inertial microfluidic fractional are as described herein.

In certain embodiments, the T cells product comprises at least 60%, at least 70%, or at least 80% viable T cells.

In certain embodiments, the T cells (CD3+) comprise CAR-T cells, memory T cells and/or central memory T cells.

T cells and method for identifying T cells are known in the art. In certain embodiments, the T cells comprise CD8+ T cells. In certain embodiments, the T cells comprise CD4+ T cells.

In certain embodiments, the T cells comprise human T cells. The use of non-human T cells in the product is also contemplated.

In certain embodiments, the T cells comprises cells that have been frozen and thawed.

In certain embodiments, the T cell product comprises an isotonic medium. Examples of media for use for infusion of T cells are known in the art, and described herein.

In certain embodiments, the non-viable cells comprise non-viable T cells. Methods for identifying non-viable cells are known in the art.

In certain embodiments, the T cell product comprises 1×10⁶/ml to 1×10⁸/ml T cells. In certain embodiments, the T cell product comprises at least 10⁷ cells, or at least 10⁸ cells.

In certain embodiments, the T cell product comprises at least 1×10⁶/ml T cells. In certain embodiments, the T cell product comprises 1×10⁶/ml to 5×10⁶/ml, 1×10⁶/ml to 1×10⁷/ml, 1×10⁶/ml to 2×10⁷/ml, 1×10⁶/ml to 3×10⁷/ml, 5×10⁶/ml to 1×10⁷/ml, 5×10⁶/ml to 2×10⁷/ml, 5×10⁶/ml to 3×10⁷/ml, 1×10⁷/ml to 2×10⁷/ml, 1×10⁷/ml to 3×10⁷/ml or 2×10⁷/ml to 3×10⁷/ml T cells.

In certain embodiments, the T cell product comprises 1×10⁶ to 1×10⁹ T cells.

In certain embodiments, the T cell product comprises at least 1×10⁶ T cells. In certain embodiments, the T cell product comprises 1×10⁶ to 5×10⁶, 1×10⁶ to 1×10⁷, 1×10⁶ to 2×10⁷, 1×10⁶ to 3×10⁷, 5×10⁶ to 1×10⁷, 5×10⁶ to 2×10⁷, 5×10⁶ to 3×10⁷, 1×10⁷ to 2×10⁷, 1×10⁷ to 3×10⁷, or 2×10⁷ to 3×10⁷ T cells.

In certain embodiments, the T cell product is depleted in non-viable cells. In certain embodiments, the T cell product comprises less than 20%, less than 15%, or less than 10%, of non-viable cells.

In certain embodiments, the T cell product is depleted for T-regulatory and/or T-suppressor cells.

In certain embodiments, the T cell product comprises less than 2%, less than 1.5%, or less than 1% one more undesired constituents, such as DMSO or cell debris.

In certain embodiments, the one or more undesired constituents comprises DMSO.

Certain embodiments of the present disclosure provide a method of depleting non-viable cells from a mixture comprising T cells, the method comprising subjecting the mixture comprising the T cells to inertial microfluidic fractionation and obtaining a fraction enriched for T cells and depleted in non-viable cells.

Methods for depleting non-viable cells from T cells are as described herein.

Certain embodiments of the present disclosure provide T cells depleted of non-viable cells produced by a method as described herein.

Certain embodiments of the present disclosure provide a method of depleting one or more undesired constituents from a T cell mixture, the method comprising subjecting a mixture comprising T cells and one or more undesired constituents to inertial microfluidic fractionation and obtaining a fraction comprising viable T cells and depleted in one or more of the undesired constituents.

Methods for depleting one or more constituents from a T cell mixture are as described herein.

Certain embodiments of the present disclosure provide T cells depleted of one or more undesired constituents produced by a method as described herein.

Certain embodiments of the present disclosure provide a method of improving a transfection characteristic(s) of a T cell population, the method comprising purifying T cells present in a medium by subjecting the medium to inertial microfluidic fractionation and purifying the T cells present in a fraction of the medium, thereby improving the transfection characteristic(s) of the T cell population.

Methods for assessing the transfection characteristics of cells are known in the art.

Certain embodiments of the present disclosure provide T cells with an improved transfection characteristic produced by a method as described herein.

Certain embodiments of the present disclosure provide a T cell product comprising at least 50% viable T cells and a DMSO concentration of less than 2%.

In certain embodiments, the T cell product comprises at least 55%, at least 60%, at least 65%, at least 70%, at least 75%, or at least 80% viable T cells.

In certain embodiments, the DMSO concentration is less than 1%.

Certain embodiments of the present disclosure provide an inertial spiral microfluidic fractionation device comprising at least two inlets and a plurality of outlets, the device comprising a channel having height of 50 to 150 μm.

Methods for producing devices for use in inertial spiral microfluidic fractionation are known in the art.

Certain embodiments of the present disclosure provide use of a device as described herein for purifying T cells.

Methods for using inertial spiral microfluidic fractionation devices for purifying T cells are described herein.

Standard techniques may be used for cell culture, molecular biology, recombinant DNA technology, tissue culture and transfection. The foregoing techniques and other procedures may be generally performed according to conventional methods well known in the art and as described in various general and more specific references that are cited and discussed throughout the present specification. See e.g., Molecular Cloning: A Laboratory Manual, 3rd ed., Vols 1,2 and 3, J. F. Sambrook and D. W. Russell, ed., Cold Spring Harbor Laboratory Press, 2001; herein incorporated by reference.

The present disclosure is further described by the following examples. It is to be understood that the following description is for the purpose of describing particular embodiments only and is not intended to be limiting with respect to the above description.

### Example 1—Inertial Microfluidic Purification of CAR-T Cell Products

Abstract

CAR-T cell therapy is rapidly emerging cancer therapy, especially in blood cancers. However, the CAR-T cell manufacturing process is complex and time, labour and cost intensive, and suffers from significant problems and a lack of standardization. A major issue is the large variability in the quality of the final CAR-T-cell product in terms of viability, functionality and phenotype composition. For example, a substantial number of CAR-T cell are rejected due to failure to meet the 80% viability specification set by the FDA for commercial products. This not only incur significant costs for the manufacturer but is also detrimental to patients who cannot receive their scheduled treatment. We demonstrate here that inertial microfluidic devices efficiently remove non-viable cells while at the same time reducing the cryoprotectant DMSO concentration in a CAR-T cell product. A spiral inertial microfluidic could deplete within minute 72% (SD±0.17) and 77% (SD±0.19) of dead cells at a 62% (SD±0.15) and 77% (SD±0.02) recovery of viable cells for CAR-T cells and untransduced T cells respectively, yielding significant increase in the overall viability rates. Up to 91% of DMSO was removed for CAR-T cells formulated in CryoStore™. Importantly, the purification process did not impact of the proliferation rates and cytotoxicity of the CAR-T cells. Interestingly, −50% of T-regulatory and T-suppressor cells as well as −55% of terminally differentiated effector cells were depleted in the final products, suggesting the potential for the inertial microfluidic process to improve the phenotypical composition of the CAR-T cell products.

### 2. MATERIALS AND METHODS

2.1 CAR-T and Untransduced T-Cells

Human CAR-T and untransduced T-cells were provided by Carina Biotech. The cryovials were transferred on dry ice and stored at −80° C./liquid nitrogen until used. Thawing was done quickly with gentle agitation in a 37° C. water bath. The cells were then either washed and resuspended in PBS+1% FBS at a concentration of 7×10⁶ cells/mL for initial optimizations or introduced as is (thawed in Cryostor) into the device through the cell suspension inlet.

2.2 Fabrication of the Microfluidic Device

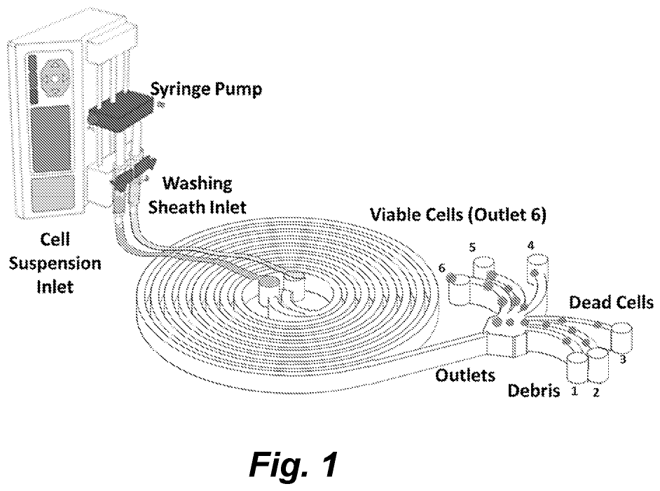

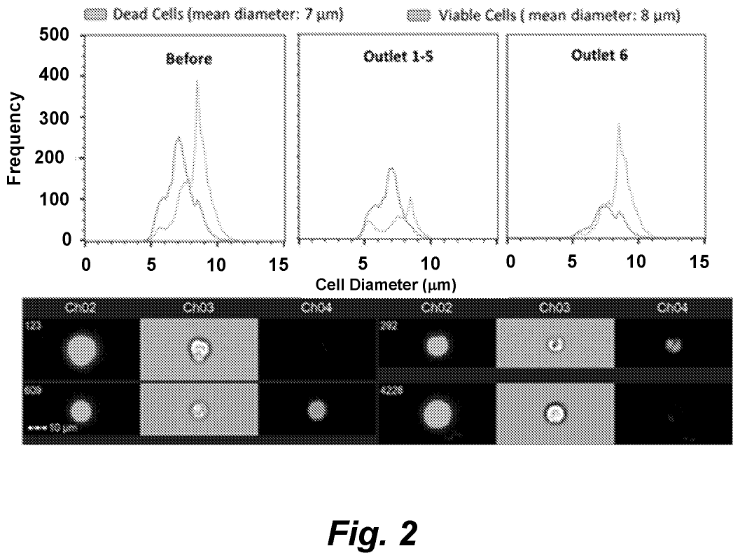

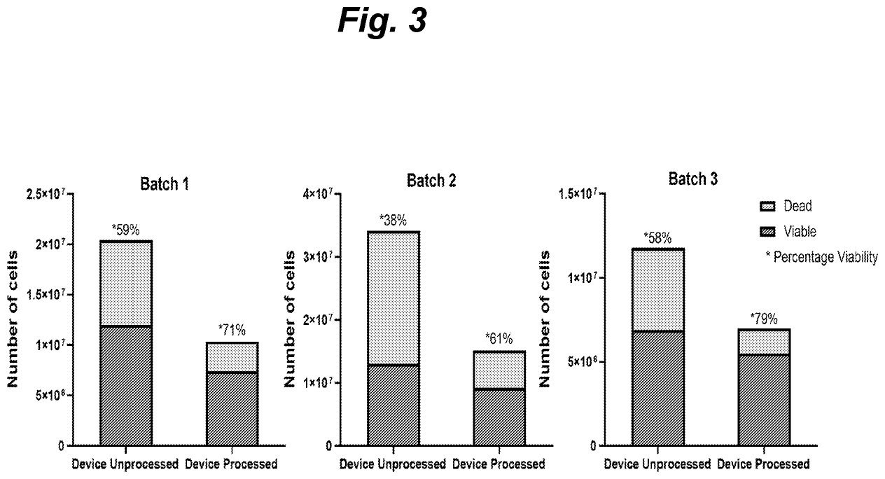

An eight-loop polydimethylsiloxane (PDMS) spiral microchannel device (inner radius=0.8 cm, outer radius=1.8 cm, channel height=130 um, width 500 um) was designed, comprising two inlets and six outlets as shown in FIG. 1. The sample inlet was used to inject the cellular suspension while the sheath inlet was used to inject the sheath solution. The SU-8 mould for casting the PDMS device was fabricated by photolithography at the Australian National Fabrication Facility (ANFF University of South Australia, Adelaide). The PDMS devices were prepared by pouring a mixture of the base and curing agent (Sylgard 184, Dow Corning Inc.) in a 10:1 ratio into the SU-8 mould, degassing and then curing at 60° C. Once cured, the PDMS devices were released from the moulds and bonded to glass slides using air plasma (Harrick Plasma, USA) at a pressure of 800 Torr for 3 minutes.

2.3 Separation in Microfluidic Device

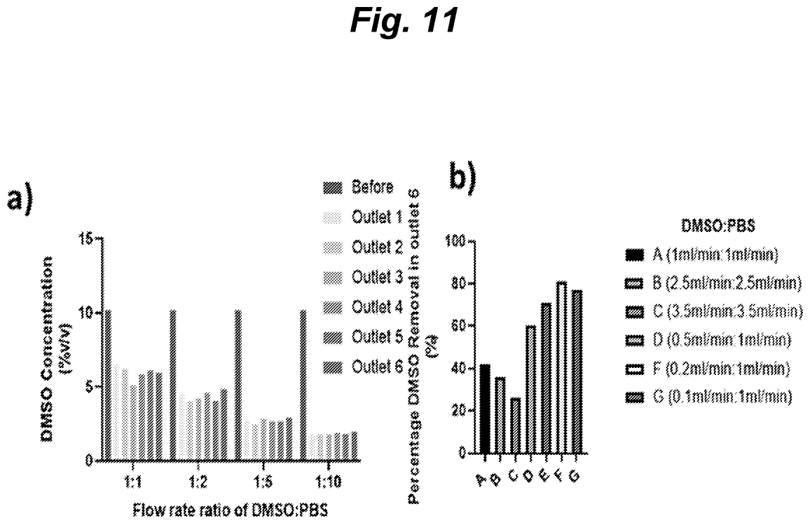

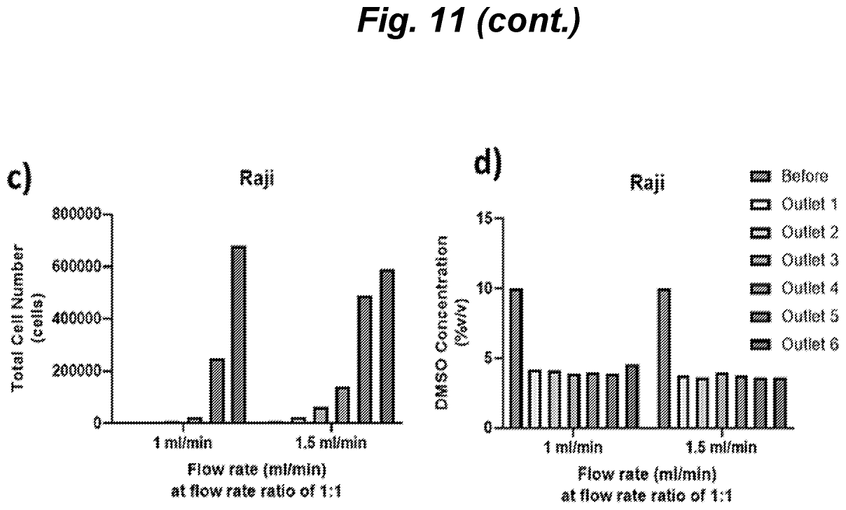

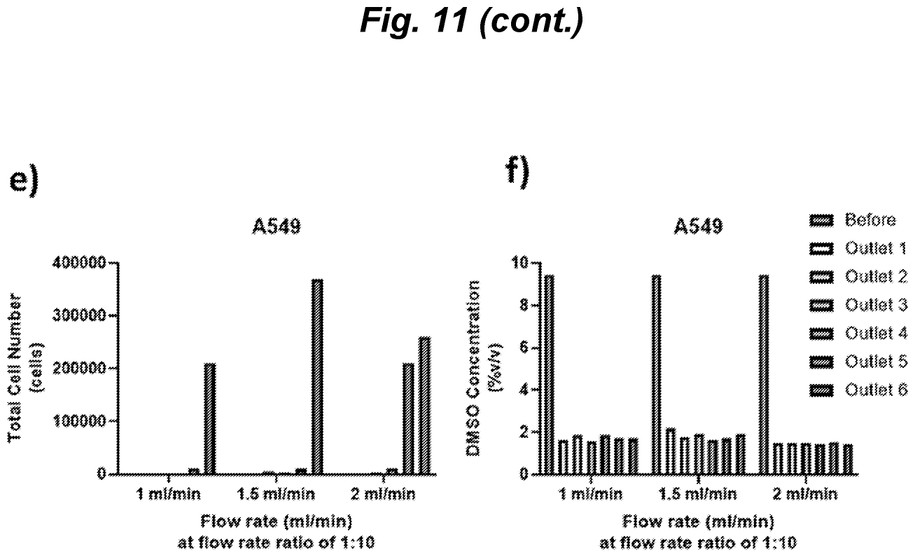

Initial optimization at different inlet flow rates was done using cell free PBS containing 10% DMSO (concentration used in typical cryopreservation protocols for hematopoietic cell products). A cell laden 10% DMSO in PBS was then first tested using mixture of the Raji cell line (CCL-86™, ATCC, USA) and A549 cell line (CCL-185™, ATCC, USA) as shown in FIG. 11. After that optimization was performed with CAR-T cell suspension in PBS+1% FBS at a cell concentration of 7×10⁶ cells/ml. The separation in the microfluidic device was done at a flow rate of 1 mL/min and fed with PBS+1% FBS from the other inlet, set at the same flow rate (1:1 flow rate ratio) as shown in the schematic representation in FIG. 1. The enriched viable cells fraction is collected in the inner outlet (outlet 6) (associated to larger cells), while dead cells and debris are collected in outer outlets of the device. Further optimization was performed using cell suspensions in Cryostor at the same cell concentration as thawed and were separated in the microfluidic device at a flow rate of 1 mL/min using plasmalyte+5% BSA as the sheath injected in the other inlet set at the same flow rate. Different flow rates and flow rate ratios for the two inlets were then tested to optimize the device performance. Separated cells were collected in the outlets of the device. The volumes were measured, and cells collected by centrifugation and analysed for viability and phenotypes. The percentage of DMSO cryoprotectant was measured before and after running in the device.

2.4 Cell Viability Measurements and Cell Size Distributions

The viability of the cell suspensions was counted manually using a trypan blue exclusion assay on a haemocytometer. Cell size distributions were determined by staining for CD3 using anti-human CD3 FITC (clone SK7) (Life Technologies, Australia) and Propidium Iodide (PI) exclusion for gating live versus dead cells. Analyses were carried out using an Imaging flow cytometer ImageStreamx Mark II (AMNIS, Seattle, WA, USA). Analysis of the cellular populations was performed with the IDEAS software Version 6.1 (AMNIS, Seattle, WA, USA) and FlowJo V10 (FLOWJO, USA). Cell size distributions were calculated using an adaptive erode mask (M04 CH04 77) on the AMNIS software, which allows an accurate calculation of cell diameters.

2.5 CAR-T Cell Phenotyping

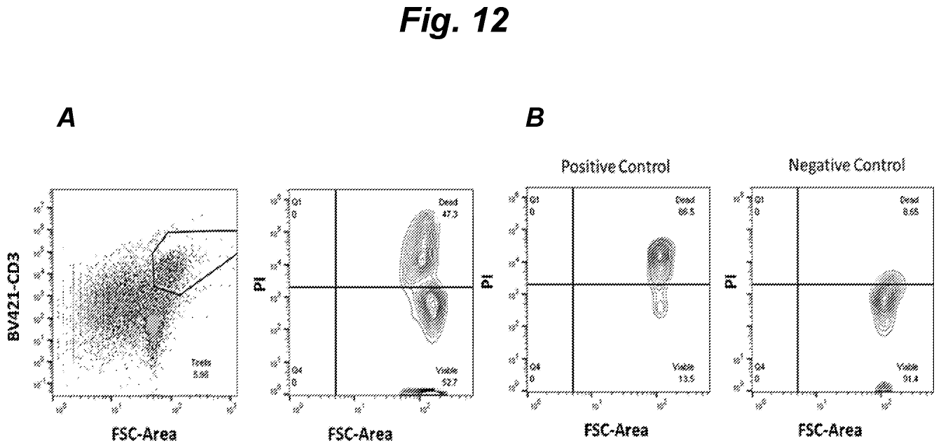

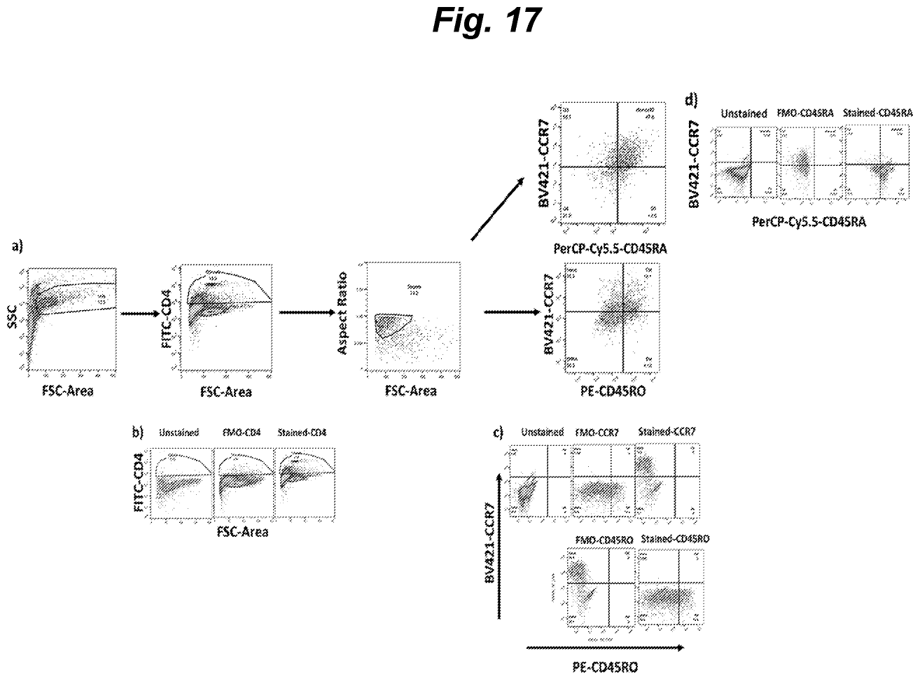

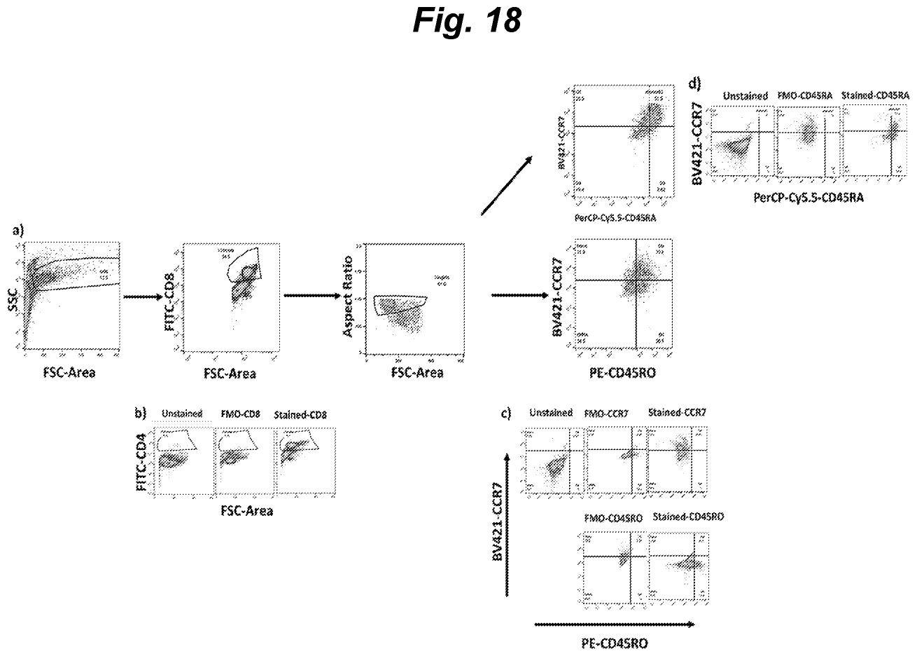

Cells were stained for surface markers and visualized using an imaging flow cytometer: ImageStreamx Mark II (AMNIS, Seattle, WA, USA). T-cells were stained with anti-human CD3 FITC (clone SK7) or anti-human CD3 BV421 (clone UCHT1) and phenotypes were characterised using anti-human CD45RO PE (clone UCHL1), CD4 EF450 (clone SK3), CD8A PECYN5.5 (clone RPA-T8), CD45RA PerCP-CY 5.5 (clone HI 100), (Life Technologies, Australia) and CCR7 BV421 (clone 150503) (BD Life sciences, Australia), CD25 PE-Cy7 (clone M-A251) (BD Life sciences, Australia) and CD4 FITC (clone OKT4) (Biolegend, Australia) The cells were also stained with FOXP3 Alexa Fluor-647 (Biolegend, Australia) after fixation and permeabilization using the FOXP3 fixation and permeabilization buffer set (Biolegend, Australia) as per the manufacturer's instructions. The gating strategy and controls used are shown in the FIGS. 12, 16, 17, 18,19.

2.6 DMSO Measurements Before and After Device Separation Using UV-Vis Spectrophotometer and Capillary Electrophoresis

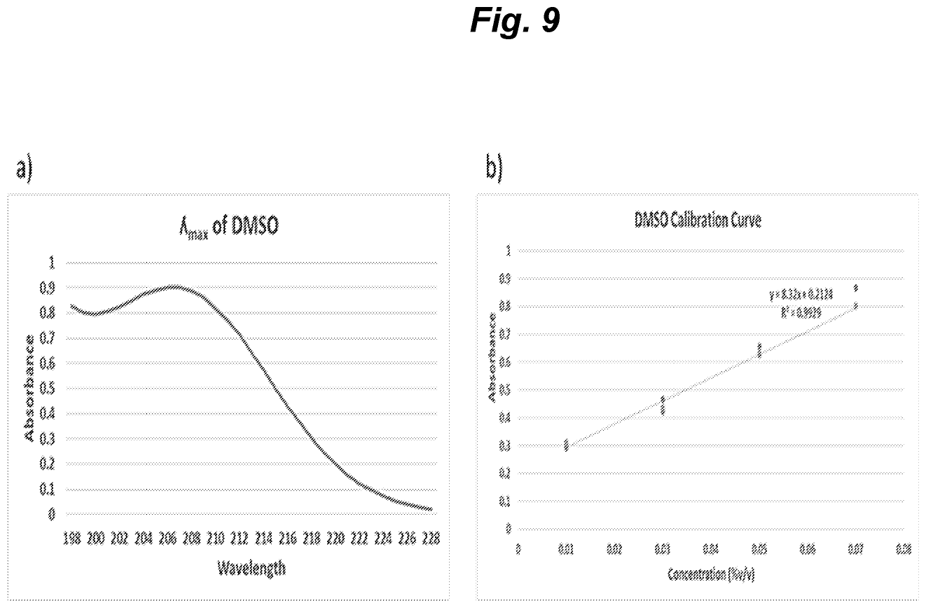

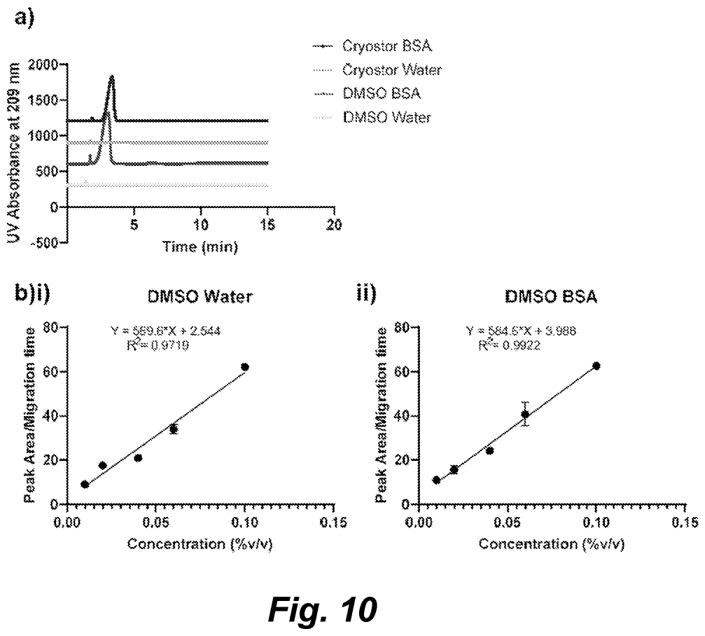

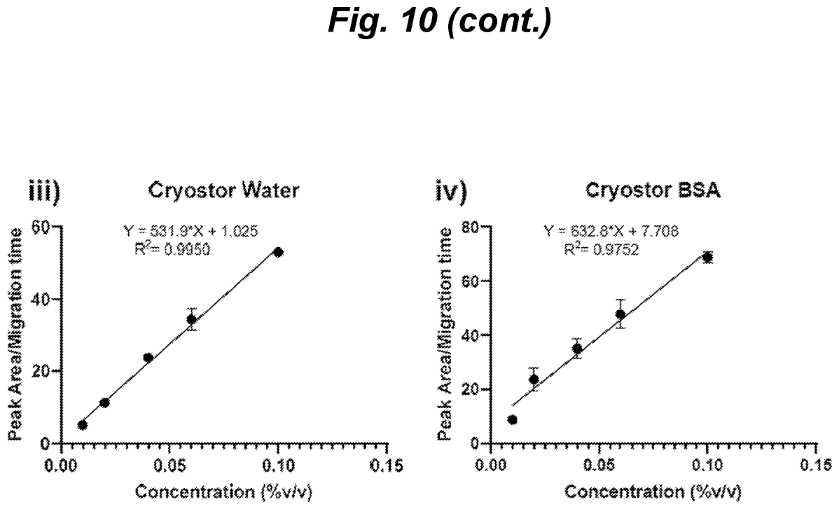

DMSO concentrations before and after device separation was determined initially by UV-Vis spectrophotometry using the Thermo Evolution 201 UV-VIS spectrophotometer (Thermofisher, Australia), according to previously established methods (Mata, C, Longmire, E K, McKenna, D H, Glass, K K & Hubel, A 2008, ‘Experimental study of diffusion-based extraction from a cell suspension’, Microfluidics and Nanofluidics, vol. 5, no. 4, pp. 529-540). The λₘₐₓ for DMSO absorption was determined at 209 nm as shown in FIG. 9, corresponding with previous reports (Hanna, J, Hubel, A & Lemke, E 2012, ‘Diffusion-based extraction of DMSO from a cell suspension in a three stream, vertical microchannel’, Biotechnology and Bioengineering, vol. 109, no. 9, pp. 2316-2324). To calculate DMSO concentrations, a calibration curve at the λₘₐₓ for DMSO absorption at 209 nm was then created using serial dilutions of a DMSO stock solution at (10% v/v) diluted in PBS. It was found to be linear across a narrow concentration range from 0.01 to 0.07% v/v. Curves were calculated from three measurements. DMSO was measured after centrifugation and removal of the cells. The DMSO concentrations were serially diluted to fall in the linear range in the calibration curve and then the concentration calculated using the calibration equation. The % v/v DMSO in each outlet was calculated and the % removal. For experiments involving cells suspended in Cryostor and washing with plasmalyte+5% BSA, capillary electrophoresis was carried out to separate the DMSO from the BSA. Capillary electrophoresis was used for experiments involving the use of plasmalyte+5% BSA as the washing sheath. BSA interferes with the DMSO reading on the UV-Vis spectrophotometer so they had to be separated using a chromatographic method. Capillary electrophoresis was selected as a suitable method and was carried out as has been cited in previous literature (Oliver, JD, Sutton, A T, Karu, N, Phillips, M, Markham, J, Peiris, P, Hilder, E F & Castignolles, P 2015, ‘Simple and robust monitoring of ethanol fermentations by capillary electrophoresis’, Biotechnology and Applied Biochemistry, vol. 62, no. 3, pp. 329-342; Pande, P G, Nellore, R V & Bhagat, H R 1992, ‘Optimization and validation of analytical conditions for bovine serum albumin using capillary electrophoresis’, Analytical Biochemistry, vol. 204, no. 1, pp. 103-106). Separations were performed on an Agilent 7100 (Agilent Technologies, Waldbronn, Germany) with a diode array detector monitoring at 209 nm which is the λₘₐₓ determined from the previous experiment. Fused-silica capillaries (50 μm i.d., 360 μm o.d.) were obtained from Polymicro (Phoenix, AZ, USA). The capillary length was 48 cm with a 39.5 cm effective length. The capillary was pre-treated prior to use by flushing with 1 M NaOH followed by water, and then the background electrolyte (BGE) for 20 Min each. The sample was injected by applying 17 mbar of pressure for 8 Sec 0 nL in 130 mM NaOH) followed by BGE, injected in the same manner. Between each run, the capillary was flushed with BGE for 10 Min. At the end of a series of injections, the capillary was flushed for 1 Min with 1 M NaOH, 10 Min with water, and 10 Min with air. The detection of the DMSO and BSA peaks using borate buffer was carried out as shown in Figure S 10a. Calibration curves of DMSO and Cryostor were generated at 209 nm using serial dilutions in the presence and absence of plasmalyte+5% BSA as shown in FIG. 10b. Integration was performed on the Agilent software (Agilent Technologies, Waldbronn, Germany) on electropherograms and corrected for the electrophoretic mobility by dividing the area of the peak by the migration time.

2.7 Cell Proliferation Assay

Cell proliferation was determined by staining with the proliferation dye Carboxyfluorescein succinimidyl ester (CFSE) (Biolegend, Australia) as described in the manufacturer's guidelines. In all cases, dye levels were titrated to achieve the brightest median fluorescence intensity, most uniform staining distribution (CV) and the best viability in culture for the cell lines used in this study. The excitation and emission wavelengths of CFSE-labelled cells were 492 nm and 517 nm respectively. Viable leukocytes were resuspended at 2×10⁶ cells/mL in PBS and labelled with 10 μM CFSE for 10 min at 37° C. The reaction was stopped by adding an equal volume of FBS and incubation for 2 min at room temperature. The cells were then washed twice, and the CFSE-labelled cells were cultured for 48 hrs at 37° C. and 5% CO2 in 96-well microtiter plates. To stimulate the proliferation of the CAR-T and untransduced cells, we activated the cells using anti CD3 coated plates (5 μg/mL) and soluble antiCD28 (4 μg/mL) and IL2 (500 U/mL). Proliferation was compared between the non-enriched and enriched cells after separation at 72 hrs and at 168 hrs. T-cells were gated for viability using CD3 staining and PI exclusion (0.1 mg/mL) and then proliferation analysis performed by the imaging flow cytometer.

2.8 Cytotoxicity Assay

The CAR-T and untransduced cells were co-cultured with the PC-3-Luc2 cancer cell line (ATCC® CRL-1435-LUC2™, ATCC, USA) at different ratios (30:1, 0.3:1 and 1:1) and cultured overnight for 16 hr in 96 well plates in complete X-vivo medium (X-Vivo 15, Lonza, Australia) supplemented with 5% inactivated human serum (Life technologies, Australia) and 1% glutamate. After coculture for 16 hrs, all luciferase assays were performed with Bright-Glo™ Luciferase Assay System (Promega, Australia), and carried out according to the manufacturer's protocol. Briefly, 100 μL of Brightglo reagent was added to cells grown in 100 μL of medium and after complete cell lysis, the 200 μL mix was transferred to an opaque 96-well plate and measured using a luminometer ( ). Each experiment was done in triplicate and the cytotoxicity percentage was calculated using the following formula:

$$\% cytotoxicity = 100 - [ \frac{( experimental cytolysis - media only )}{( maximum cytolysis - media only )} \times 100 ]$$

2.9 Statistical Analysis

All experiments were repeated at least three times and analysed non-parametrically using GraphPad Prism. The data was analysed using two-way ANOVA and a Fisher LSD multiple comparison test.

### 3. RESULTS AND DISCUSSION

3.1 Inertial Microfluidic Enrichment of Viable CAR-T Cells

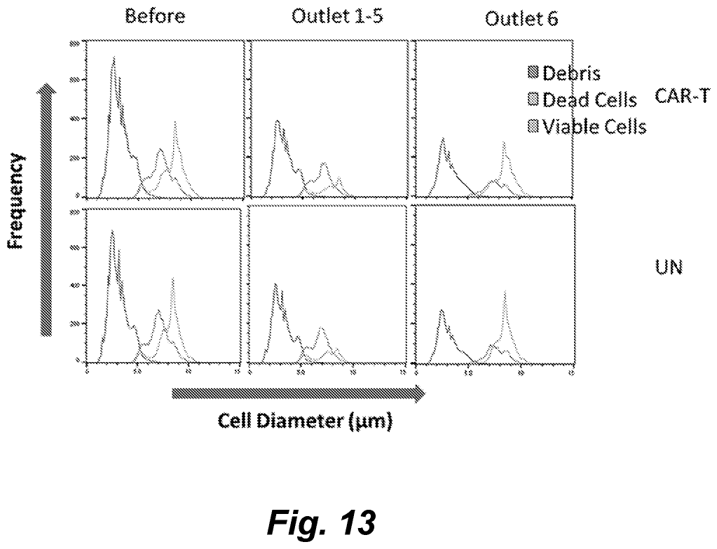

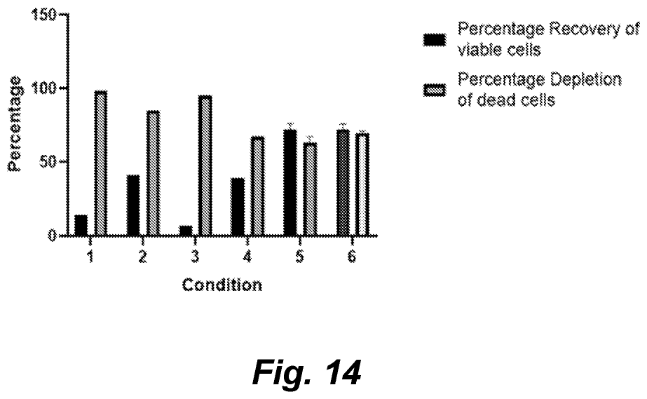

In order to investigate the potential of inertial microfluidics to separate viable from dead CAR-T cells, we first determined using imaging flow cytometry the respective size distributions in a batch of CAR-T cells. The same measurements were also performed for untransduced T cells from the same batch as shown in FIG. 13. As expected, viable cells in both the CAR-T cells and untransduced control cells presented larger mean cell diameters compared to dead cells (8 μm±0.07 vs. 7 μm±0.07, FIG. 1). This difference suggested the feasibility for effective separation of these two cell populations using inertial microfluidics. We designed and fabricated using soft lithography an inertial microfluidic spiral device adapted from previous studies (Bhagat, A A S, Kuntaegowdanahalli, S S & Papautsky, I 2008, ‘Continuous particle separation in spiral microchannels using dean flows and differential migration’, Lab on a Chip, vol. 8, no. 11, pp. 1906-1914; Warkiani, M E, Khoo, B L, Tan, D S-W, Bhagat, A A S, Lim, W-T, Yap, Y S, Lee, S C, Soo, R A, Han, J & Lim, C T 2014, ‘An ultra-high-throughput spiral microfluidic biochip for the enrichment of circulating tumor cells’, Analyst, vol. 139, no. 13, pp. 3245-3255; Ye, X, Liu, H, Ding, Y, Li, H & Lu, B 2009, ‘Research on the cast molding process for high quality PDMS molds’, Microelectronic Engineering, vol. 86, The Fourth IEEE International Symposium on Advanced Gate Stack Technology (ISAGST 2007), no. 3, pp. 310-313.). The device comprises two inlets for respectively the cell suspension and sheath, as well as 6 outlets from which the fractionated cell suspensions can be collected. The larger cellular fraction is enriched in outlet 6 while the debris are mostly found in outlet 1. The device operation was first optimised using mixtures of various cell lines, mimicking large (viable) and smaller (dead) cells as shown in FIG. 11.

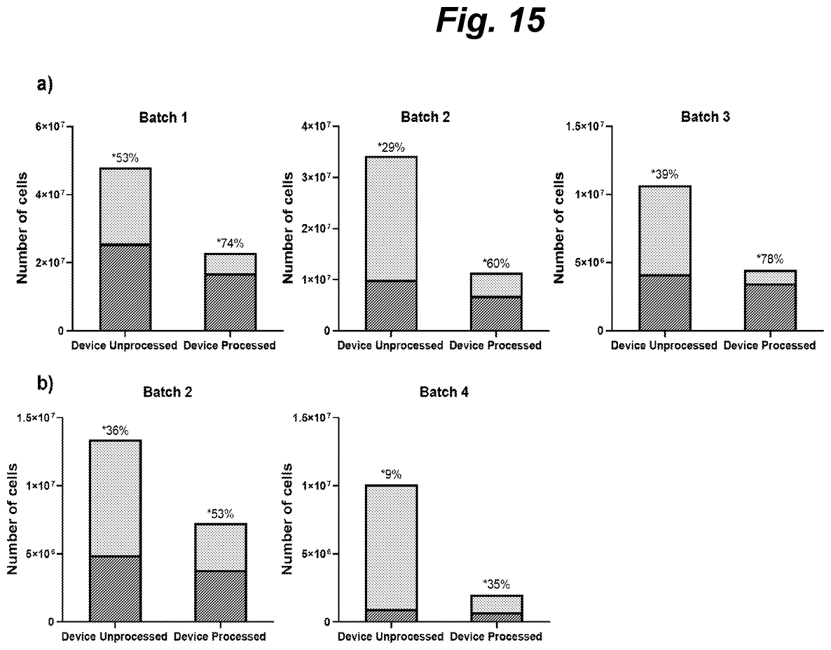

Using three different batches of CAR-T cell product and the corresponding untransduced T-cells, we next assessed the enrichment afforded from processing the cell suspensions through the inertial microfluidic devices. The cell suspensions in PBS+1% FBS (7×10⁶ cells/mL) were separated in the device with a total flow rate of 1 mL/min and using PBS+1% FBS as the sheath with an inlet flow rate ratio of 1:1 (cellular suspension: Sheath). The viability of the cells was determined before and after device separation by trypan blue staining. It is noteworthy that the various CAR-T cell and untransduced cellular batches used in the study had significantly different percentages of viable cells, with values ranging from extremely very low viability (38%) to moderate ones (58% and 59%). Processing in the spiral inertial microfluidic device yielded significant increases in the percentages of viable cells in the suspension collected from the outer outlet 6 for all tested batches. Dead cells were predominantly collected in outlets 2-5. The average percentage of viable CAR-T cells increased from 51% (SD±0.12) to 71% (SD±0.09). Importantly, the recovery of viable cells ranged from 62%-80% in the 3 tested batches. Similar data was obtained for the untransduced cells, with viability increases from 40 (SD±0.12) to 71% (SD±0.09) and recovery of viable cell ranging from 66%-84% (FIG. 15a). Additional viability measurements were conducted for the CAR-T cell batch 3 through staining for CD3 and PI, yielding similar percentage increase and recoveries of viable cells than those obtained with trypan blue staining.

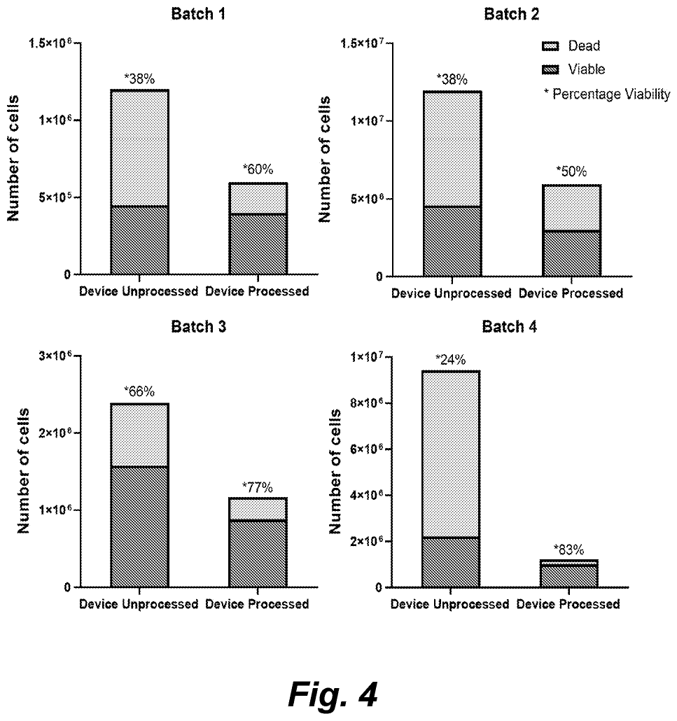

Following on these promising initial results, we next investigated the performance of inertial microfluidic processing in purifying CAR-T cell products in more clinically relevant solutions. In a common clinical scenario, CAR-T products are frozen at high cell concentration and stored in a cryoprotectant such as Cryostor. The CAR-T cells are then diluted with the infusion agent plasmalyte+5% BSA prior to being administrated to patients. Using plasmalyte+5% BSA as the sheath and CAR-T cells suspended in Cryostor at very high concentration (7×10⁷ cells/mL), we mimic point of care purification of a CAR-T cell product prior to administration to the patient. However, using the operating conditions used above (flow rate inlet ratio of 1:1), we achieved a 98% depletion of non-viable cells, but the recovery of viable cells was very low (14%). This significant difference in performance was likely due to the large difference in viscosities between Cryostor and plasmalyte+5% BSA, which prevented efficient mixing of the two inlet streams in the device. We thus tested several conditions, using different flow rate ratios, inlet flow rates and length outlets (used to modulate the pressure inside the device) as summarized in FIG. 24. Using a higher inlet flow rate ratio of 1:10, a good separation efficiency was achieved, similar to the level of performance previously measured for cells suspended in PBS+1% FBS and using a 1:1 inlet flow rate ratio. Under these reoptimized conditions, 72% (SD±0.17) and 77% (SD±0.19) depletion of dead cells were achieved for the CAR-T cells and untransduced cells, respectively. The recoveries of viable cells were 62% (SD±0.15) and 77% (SD±0.02) respectively. This resulted in an increase in the average percentage of viable cells from 34% (SD±0.07) to 62% (SD±0.15) for CAR-T cell product and 27% (SD±0.17) to 44% (SD±0.13) for untransduced batches, respectively. The data for each of the 4 individual CAR-T cells batch is shown in FIG. 4. The data for untransduced cells is shown in FIG. 15b.

It should be noted that the concentration of the cell suspension is a key factor in the inertial microfluidic process and high concentration significantly decrease separation efficiencies due to increased cell-cell interactions which lead to the defocusing of the streams and a reduction in inertial focusing efficiency. The high CAR-T cells concentration used in the clinical scenario above might contribute to the poor recovery of the viable cells when processed using a 1:1 inlet flow rate ratio. However, under the 1:10 inlet flow rate ratio, the rapid mixing occurring in the device effectively dilutes the cell suspension.

3.2 Inertial Microfluidic Processing Efficiently Reduces Cryoprotectant in CAR-T Cell Products

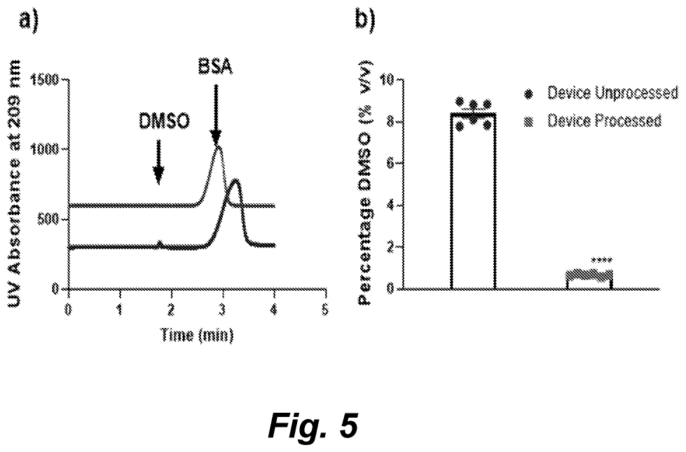

In order to test the capability of the device to remove DMSO used as model cryoprotectant, a calibration curve was first done using UV-Vis spectrophotometry, then the concentration of the DMSO before and after the device in the CART cell collection outlet (outlet 6) was measured. Increasing the overall flow rate from 1 mL/min to 2.5 mL/min and 3.5 mL/min at an inlet flow rate ratio of 1:1, tended to reduce the DMSO removal capability of the device (data shown in FIG. 11b; respectively conditions A, B, C). In contrast, increasing the inlet flow rate ratio increased the percentage of DMSO removal significantly. A flow rate ratio of 1:1, 1:2, 1:5 and 1:10 reduced DMSO by 40%, 60%, 70% and 90% respectively (condition A vs. D vs. F vs. G in FIG. 11b). However, this is also associated with an increase in the total volume collected. For flow rate ratios of 1:1, 1:2, 1:5 and 1:10, if 1 mL of cells in cryoprotectant is processed, the resulting volumes will be 0.75, 1, 2 and 4 ml respectively. When these conditions were tested with Cryostor in one inlet and the washing sheath of plasmalyte+5% BSA was tested it was found that consistent mixing and cell focusing occurred only at flow rate ratio of 1:10 due to the difference in viscosities between the thawed Cryostor cell suspension and the plasmalyte+5% BSA. At the flow rate ratio of 1:10, 92% (SD±0.01) of DMSO was removed as shown in FIG. 5b.

3.3 Inertial Microfluidic Processing does not Affect the Percentage of Transduced CAR-T Cells

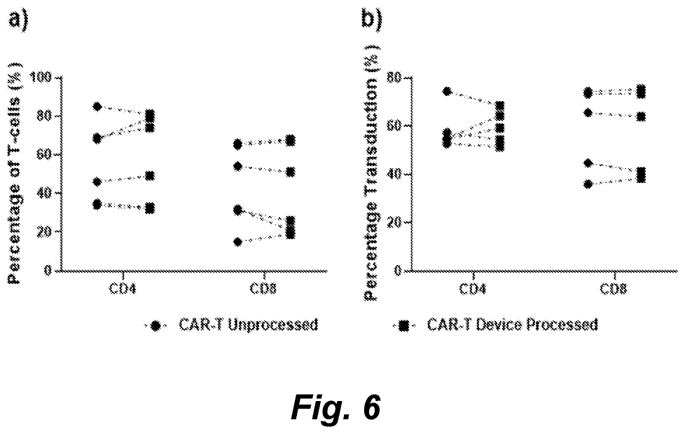

Having identified optimal operating conditions yielding efficient enrichment of the percentage of viable cells and significant reduction in the amount of cryoprotectant, we next endeavour to verify that no depletion of transduced CAR-T cells occurred. To this end, the percentage transduced CART cells was assessed before and after processing in the inertial microfluidic device. No significant changes were measured for both CD4 (59% (SD±0.09) vs 60% (SD±0.07)) and CD8 (59% (SD±0.17) vs 58% (SD±0.18)) CAR-T cells, as shown in FIG. 6a.

3.4 Inertial Microfluidic Processing Enriches Central Memory and Stem Cell Phenotypes and Depletes T-Regulatory and T-Suppressor Cells

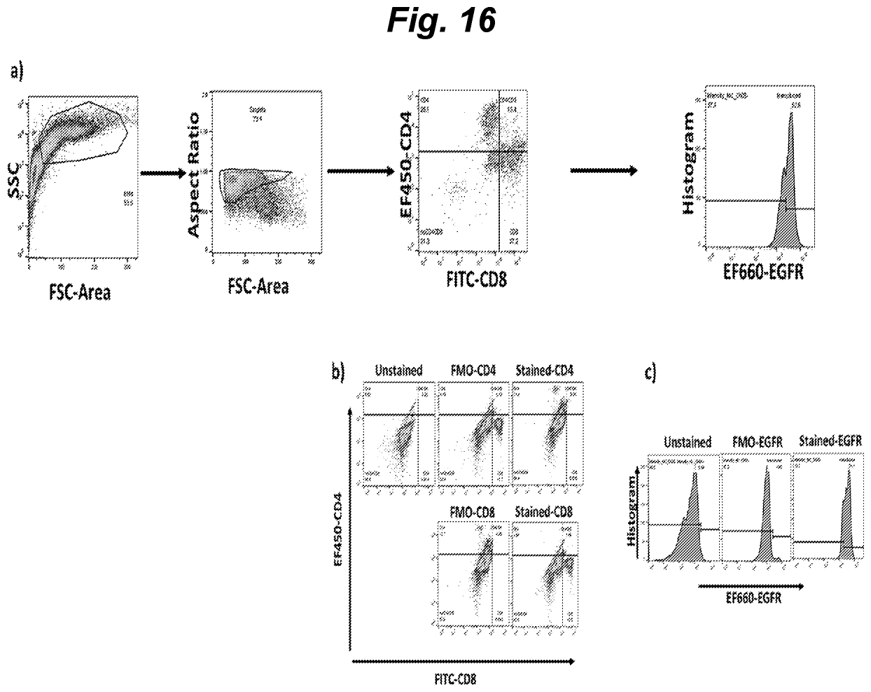

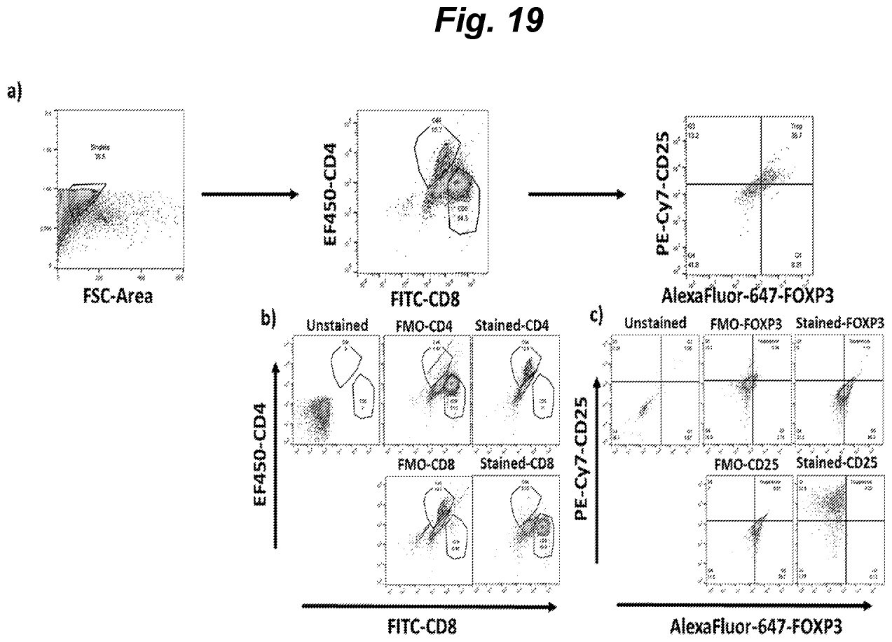

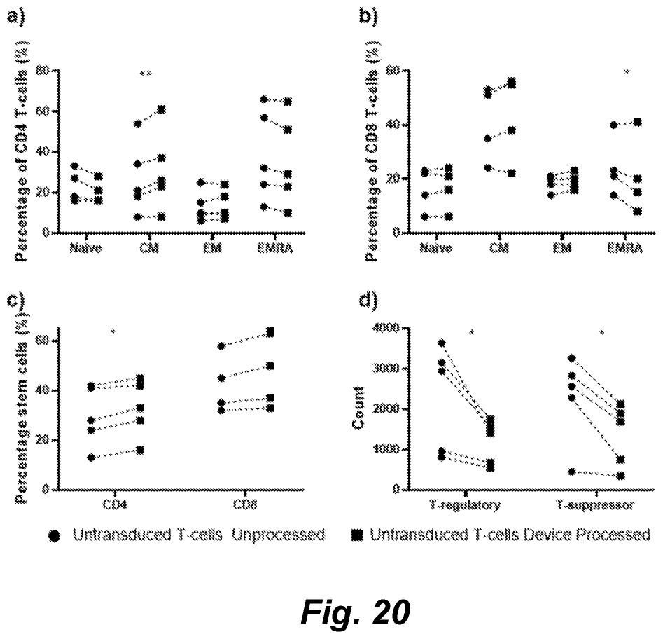

We next investigated the phenotypic characteristics of the purified CAR-T cells products as compared to the product before processing. It is well reported now that the phenotypic properties of the cells are related to T-cell longevity and persistence inside the body after transfusion. It has been shown that naïve T-cells and central memory T-cells are the longer-lived ones and their initial percentage in the transfused product is directly linked with treatment outcomes and prognosis. This has led newer studies to make a modification in the ex vivo expansion media by adding IL7 and IL15 homeostatic cytokines or other chemicals to increase such phenotypes and prevent differentiation to the terminally differentiated cells. In other studies they have increased the central memory phenotype by genetically modifying the cells through knocking out the TET2 gene that affects the production of central memory cells downstream. Others have selected the particular phenotype before genetic modification and expansion by magnetic selection and Fluorescence activated sorting (FACS). The phenotypes investigated were the CD4 helper T-cells, CD8 cytotoxic T-cells, central memory markers (CD45RO, CCR7), stem cell marker (CD45RA) and markers for T-regulatory cells and T-suppressor cells (CD25+high, FOXP3+, CD127 low). Gating strategies and FMO controls are shown in FIGS. 16, 17, 18 and 19.

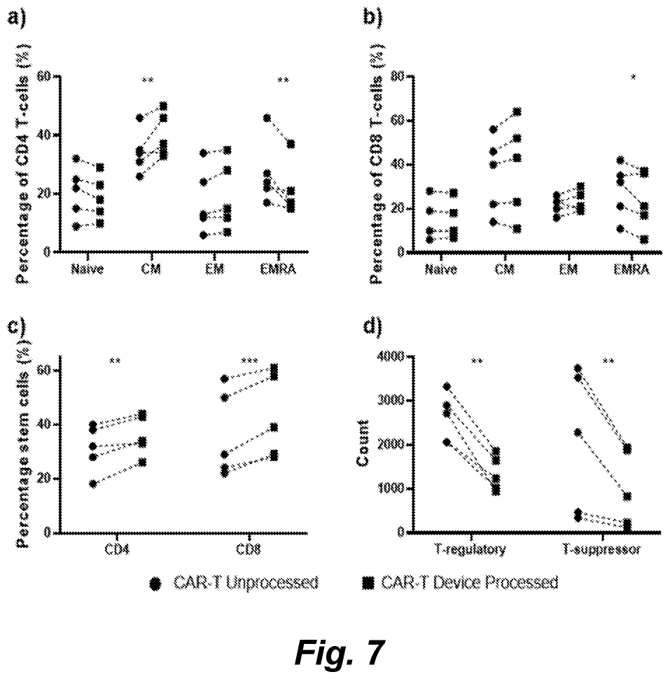

The CAR T cells batches tested here had an initial percentage of CD4 cells of 56% (SD±0.2) and 44% (SD±0.2) for CD8. There was no significant difference in the percentages of CD4 and CD8 cells before and after processing as shown in FIG. 5b. Similar results were obtained for the untransduced cells (FIG. 20). Analyses of the CD4 phenotypes indicated that there was a 57% (SD±0.1) depletion of CD4 EMRAS for the CAR-T cells. There was also a small but consistent increase in the central memory (CM) phenotype in all batches from 32% (SD±0.04 to 38% (SD±0.18) for CAR-T cells respectively. These differences in CD4 EMRAs statistically significant in both CAR-T and untransduced cells batched (n=4) and similarly for the CM cells in the untransduced (p<0.05, FIG. 7a and FIG. 20a). For CD8 cells, there was a 55% (SD±0.09) depletion of CD8 EMRAS for the CAR-T cells. This resulted in a statistically significant decrease of CD8 EMRAs from 26% (SD±0.13) to 20% (SD±0.13) (p<0.05). There was also a noticeable small, consistent but non statistically significant increase in the CM from 41% (SD±0.14) to 46% (SD±0.17) for CAR-T in all five batches (FIG. 7b). The CD45RA marker is a stem cell phenotype marker that is expressed on both CD4 and CD8 cells. We found a small but consistent and significant increase in the percentage of cells with stem cell phenotypes across all batches upon enrichment in the spiral inertial microfluidic device. For CD4 cells, the percentage increased from 30% (SD±0.09) to 34% (SD±0.07), while for CD8 cells, the percentage increased from 40% (SD±0.15) to 47% (SD±0.15). Similar data was obtained for the untransduced cells (FIG. 20c). The most significant difference in the CAR-T cells phenotypes after inertial microfluidic processing was a depletion of CD4 T-regulatory (53%±0.11) and CD8 T-suppressor cells (54%±0.09) (FIG. 6d, p<0.05 in both cases). It has been cited in previous researches that T-regulatory and T-suppressor cells isolated from cancer patients have an immunosuppressive nature and usually lymphodepletion is done prior to treatment to reduce them and their effect. Also in some researches, T-regulatory and T-suppressor cells are depleted by magnetic selection before activation and expansion of CAR-T cells due to their influence on proliferation and the exhaustion status of the cells. Whether depleting it at the end of manufacture will add value or not as in our case still needs to be further verified.

These differences in the proportions of the various T cell phenotypes can be explained by their average dimensions as determined using imaging flow cytometry. The CM (7.6 μm SD±0.9) and stem cell (8 μm SD±0.5) phenotypes had slightly larger diameters compared to other T-cells, while the T-regulatory (6.8 μm SD±0.3), T-suppressors (7.6 μm SD±1.1) and EMRAs (7 μm SD±0.2) phenotypes had lower diameters.

3.5 Inertial Microfluidic Processing of CART Cell Product has No Detrimental Effects on their Proliferation Capability and Cytotoxicity Functions

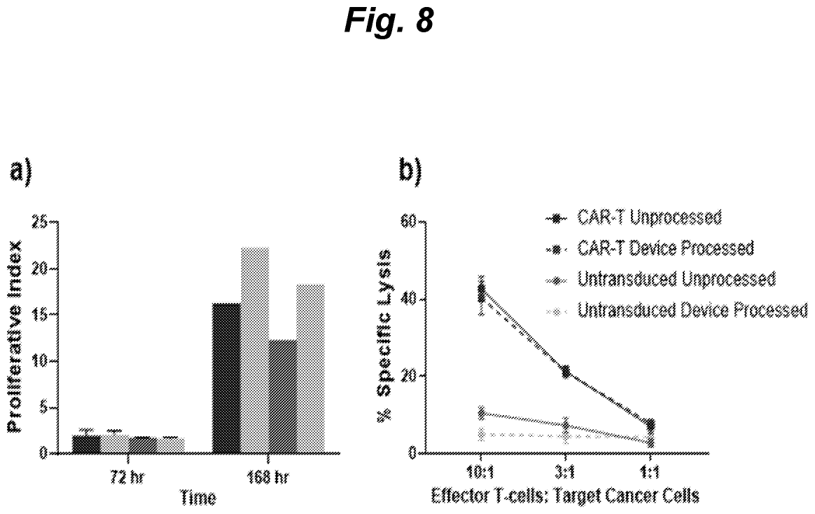

We tested the proliferation and cytotoxicity of the CAR-T cells after processing and compared it to the unprocessed cells. The same number of viable cells of processed and unprocessed cells were used in these experiments and compared. No differences in proliferation were measured as shown in FIG. 8a. The proliferative index at 72 hrs was 2.1 (SD±0.6) for processed CAR-T cells compared to 1.7 (SD±0.2) for the non-processed ones. Similar results were obtained for the untransduced cells. The cytotoxic activity of the CAR T cells was assessed against the PC-3-Luc2 cancer cell line. There was no statistically significant difference in lytic activity between CAR-T cells processed or not (p>0.05). However, a modest but statistically significant increased lytic activity was measured for CAR-T cells processed in inertial microfluidic devices at the 1:1 ratio (p<0.05).

### 4. CONCLUSIONS

This research demonstrates the power of spiral inertial microfluidic based cell size separation for enhancing the manufacturing quality of a CAR-T cell product. In clinical trial centres, the cells are washed and counted before infusion to the patient but for commercialized products, they are produced offsite in a central facility and then shipped frozen to the treating facility, where the cells are thawed and infused back to the patient. However, most of these CAR-T cell products do have dead cells (T-cells and other PBMCs), cryoprotectants like DMSO. The effective removal of dead cells and cryoprotectant DMSO using clinically relevant solutions and achieving cell recoveries of above 70% with no detrimental effects on the functionality of the CART cells demonstrates this combined dilution and cell focusing microfluidic strategy for use as a point of care cell washing and formulation step, before injection into the patient. The increase in viable cells at high cell recoveries indicate that our method can potentially rescue some out of specification viability products to become within the viability specification for clinical trials and commercial products. Our approach can deplete ˜80% of dead cells at a ˜80% recovery of viable ones with a resulting increase in viability of ˜25%. We have shown that this may lead to enhance the viability of a CAR-T cell product above the set specifications however for very low viability batches there will be an enhancement, but the set specification may not be met. Additionally, the 50% depletion of T-regulatory and T-suppressor cells and 55% of the terminally differentiated effector cells, with a small enrichment of central memory and stem cell phenotype indicate that our device may influence the resulting phenotypes with the enrichment of the more in vivo longer living phenotypes as opposed to phenotypes that may be expressing immunosuppressive receptors as seen with T-regulatory and T-suppressor cells isolated from most cancer patients.

One limitation for microfluidics systems is the limited throughput. To compensate for higher cell concentration and increased viscosities of solutions by adjustment of flow rate ratio, the throughput (processing volumes and times) were adapted but still fit. As such processing of 1 mL of formulated CAR-T product takes in 1 minute for the 1:1 flow rate ratio and results in a volume of 0.75 mL of purified product or 10 minutes for the flow rate ratio of 1:10 and results in a volume of 3 mL, while achieving an increase of viability of more than 20%. There is the potential for using multiple parallel devices with proven success to increase throughput and reduce the processing time by more than ten times.

The removal of dead cells also provides scope for its potential as a post-electroporation clean-up step, which can be integrated into a closed GMP compliant cell enrichment device. Electroporation is a well utilised approach for genetic modification by non-viral methods, which has become more popular in clinical trials to replace viral transduction in certain CAR-T cell therapies. However, electroporation is associated very low cell viability (about 40%) due to the effects of the electrical pulse. These dead cells can influence the transduction efficiency and the expansion of the other cells. The latter happens through a process called compensatory proliferation, which affects the proliferation and differentiation of stem cells and phenotypes produced. Additionally, as transduced cells are larger in cell size, a post-electroporation separation step may be additionally beneficial for removal of dead cells and increased recovery of transduced cells. Further assessment for this application is ongoing.

In conclusion, it is evident that our approach using the inertial microfluidic device can in fact be used to enhance the viability of a CAR-T cell product. It may also enhance the resulting phenotypes but may require further optimizations for that purpose. In addition, we must note that whether the enrichment of certain phenotypes over others will have an added value still needs further investigation.

Although the present disclosure has been described with reference to particular embodiments, it will be appreciated that the disclosure may be embodied in many other forms. It will also be appreciated that the disclosure described herein is susceptible to variations and modifications other than those specifically described. It is to be understood that the disclosure includes all such variations and modifications. The disclosure also includes all of the steps, features, compositions and compounds referred to, or indicated in this specification, individually or collectively, and any and all combinations of any two or more of the steps or features.

Also, it is to be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The term “about” or “approximately” means an acceptable error for a particular value, which depends in part on how the value is measured or determined. In certain embodiments, “about” can mean one or more standard deviations. When the antecedent term “about” is applied to a recited range or value it denotes an approximation within the deviation in the range or value known or expected in the art from the measurements method. For removal of doubt, it shall be understood that any range or value stated herein that does not specifically recite the term “about” before the range or before any value within the stated range inherently includes such term to encompass the approximation within the deviation noted above.

Reference to any prior art in this specification is not, and should not be taken as, an acknowledgment or any form of suggestion that this prior art forms part of the common general knowledge in any country.

The subject headings used herein are included only for the ease of reference of the reader and should not be used to limit the subject matter found throughout the disclosure or the claims. The subject headings should not be used in construing the scope of the claims or the claim limitations.

The description provided herein is in relation to several embodiments which may share common characteristics and features. It is to be understood that one or more features of one embodiment may be combinable with one or more features of the other embodiments. In addition, a single feature or combination of features of the embodiments may constitute additional embodiments.

All methods described herein can be performed in any suitable order unless indicated otherwise herein or clearly contradicted by context. The use of any and all examples, or exemplary language (e.g., “such as”) provided herein, is intended merely to better illuminate the example embodiments and does not pose a limitation on the scope of the claimed invention unless otherwise claimed. No language in the specification should be construed as indicating any non-claimed element as essential.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

1. A method of purifying T cells, the method comprising subjecting a medium comprising the T cells to inertial microfluidic fractionation and obtaining a fraction comprising purified T cells.

2. The method according to claim 1, wherein the method comprises fractionating the T cells from non-viable cells and/or one or more undesired constituents present in the medium.

3. The method according to claim 1 or 2, wherein the inertial microfluidic fractionation comprises spiral inertial microfluidic fractionation.

4. The method according to any one of claims 1 to 3, wherein the inertial microfluidic fractionation comprises an inlet ratio of the medium to a sheath medium of at least 1:1.

5. The method according to claim 4, wherein the inertial microfluidic fractionation comprises an inlet ratio of the medium to the sheath medium of approximately 1:10.

6. The method according to any one of claims 1 to 5, wherein the T cells comprise CAR-T cells.

7. The method according to any one of claims 1 to 6, whether the method comprises recovery of at least 50% of viable T cells present in the medium.

8. The method according to any one of claims 1 to 7, wherein the method depletes greater than 70% of non-viable cells present in the medium.

9. The method according to any one of claims 1 to 8, wherein the method further depletes at least 50% of T-regulatory and/or T-suppressor cells present in the medium.

10. The method according to any one of claims 1 to 9, wherein the concentration of one or more undesired constituents in the medium is reduced by at least 40%.

11. The method according to any one of claims 2 to 10, wherein the one or more undesired constituents comprises DMSO.

12. The method according to any one of claims 1 to 11, wherein the inertial microfluidic fractionation utilises a flow rate of at least 1 ml/min.

13. The method according to any one of claims 1 to 12, wherein the medium comprises at least 1×10⁶ T cells/ml.

14. A method of purifying T cells, the method comprising purifying the T cells from non-viable cells and/or one or more undesired constituents present in a medium by subjecting the medium to inertial microfluidic fractionation and obtaining a fraction comprising purified T cells.

15. T cells purified by the method according to any one of claims 1 to 14.

16. Use of T cells according to claim 15 for therapy.

17. A medicament comprising T cells according to claim 15.

18. A method of producing a T cell product, the method comprising subjecting a medium comprising the T cells to inertial microfluidic fractionation and obtaining a fraction of the medium comprising purified T cells.

19. The method according to claim 18, wherein the inertial microfluidic fractionation comprises spiral inertial fractionation

20. The method according to claim 18 or 19, wherein the inertial microfluidic fractionation comprises an inlet ratio of the medium to a sheath medium of at least 1:1.

21. The method according to claim 20, wherein the inertial microfluidic fractionation comprises an inlet ratio of the medium to the sheath medium of approximately 1:10.

22. The method according to any one of claims 18 to 21, wherein the T cells comprise CAR-T cells.

23. The method according to any one of claims 18 to 22, whether the method comprises recovery of at least 50% of T cells present in the medium.

24. The method according to any one of claims 18 to 23, wherein the method comprises purifying the T cells from non-viable cells.

25. The method according to claim 24, wherein the method depletes greater than 70% of non-viable cells present in the medium.

26. The method according to any one of claims 18 to 25, wherein the method further depletes at least 50% of T-regulatory and/or T-suppressor cells present in the medium.

27. The method according to any one of claims 18 to 26, wherein the method depletes one or more undesired constituents present in the medium.

28. The method according claim 17, wherein the concentration of one or more undesired constituents in the medium is reduced by at least 40%.

29. The method according to claim 27 or 28, wherein the one or more undesired constituents comprises DMSO.

30. The method according to any one of claims 18 to 29, wherein the inertial microfluidic fractionation utilises a flow rate of at least 1 ml/min.

31. The method according to any one of claims 18 to 30, wherein the medium comprises at least 1×10⁶ T cells/ml.

32. The method according to claim 18, wherein the T cells comprise CAR-T cells.

33. The method according to any one of claims 18 to 32, wherein the medium is a cryopreserved T cell mixture or a T cell mixture that has been expanded after cryopreservation.

34. A T cell product produced by the method according to any one of claims 18 to 33.

35. Use of a T cell product according to claim 34 for therapy.

36. A medicament comprising a T cell product according to claim 34.

37. A T cell product comprising at least 50% viable T cells, the T cell product produced by inertial microfluidic fractionation.

38. A method of depleting non-viable cells from a mixture comprising T cells, the method comprising subjecting the mixture comprising the T cells to inertial microfluidic fractionation and obtaining a fraction enriched for T cells and depleted in non-viable cells.

39. A method of depleting one or more undesired constituents from a T cell mixture, the method comprising subjecting a mixture comprising T cells and one or more undesired constituents to inertial microfluidic fractionation and obtaining a fraction comprising viable T cells and depleted in one or more of the undesired constituents.

40. A method of improving a transfection characteristic of a T cell population, the method comprising purifying T cells present in a medium by subjecting the medium to inertial microfluidic fractionation and purifying the T cells present in a fraction of the medium, thereby improving the transfection characteristic of the T cell population.

41. An inertial microfluidic fractionation device comprising at least two inlets and a plurality of outlets, the device comprising the device comprising a channel having height of X to Y μm.

42. Use of a device according to claim 41 for purifying T cells.
